(19) United States
(12) Patent Application Publication
(10) Pub. No.: US 2018/0317850 A1
(43) Pub. Date: Nov. 8, 2018

(54) STICK-ON, MULTI-ELECTRODE DEVICE FOR NEUROLOGICAL APPLICATIONS

(71) Applicant: Rhythmlink International, LLC, Columbia, SC (US)

(72) Inventors: Shawn V. Regan, Columbia, SC (US); Daniel McCoy, Murrells Inlet, SC (US); Brian Petree, West Columbia, SC (US); Harrison M. Floyd, Columbia, SC (US)

(73) Assignee: Rhythmlink International, LLC

(21) Appl. No.: 15/973,685

(22) Filed: May 8, 2018

Related U.S. Application Data
(60) Provisional application No. 62/503,085, filed on May 8, 2017.

(51) Int. Cl. A61B 5/00; A61B 5/04

(52) U.S. Cl. CPC A61B 5/68335; A61B 2562/0209; A61B 2562/043; A61B 5/04001

(57) ABSTRACT

A multi-electrode applicator includes a carrier with recesses formed in pre-selected locations on one side. Each recess carries an electrode, and optionally both an applicator and an electrode, so that, when that side of the carrier is applied to a surface, the electrodes engage the surface in their pre-selected locations. An adhesive is applied to the underside of the carrier to cause the carrier to stick to the skin surface. A release paper may be applied over the adhesive so that, when the paper is removed, the adhesive is exposed and the carrier can then be stuck to the skin. The carrier may be flat or curved, and the electrodes may be self-embedding electrodes, a stimulating- or recording-type electrode, or a ground electrode-reference electrode pair. Electrical conductor connects the electrode to a monitor directly or via a multi-pin connector.

### CROSS-REFERENCE TO RELATED APPLICATIONS

This application claims the benefit of priority of U.S. Provisional No. 62/503,085, filed May 8, 2017, which is incorporated herein by reference in its entirety.

### TECHNOLOGY FIELD

This disclosure relates to the technology field of neurological testing and monitoring, and, more specifically, to the application of electrodes for neurological signal acquisition and neurological stimulation.

### BACKGROUND

Neurological monitoring and testing involves the sensing of electrical currents in nerves or the electrical stimulation of nerves. Electrical currents traveling in nerves generate magnetic fields around the nerves that can be used to replicate the original electrical currents in conductors close enough to respond electrically to that magnetic field.

Accordingly, this phenomenon is used in neurological monitoring, and also in stimulating nerves by the reverse of the phenomenon, namely, exposing the nerves of the body to a magnetic field generated by an electrical current traveling in a conductor proximate to the nerve in the body.

In neurological monitoring of the brain, electrodes are placed in specific locations on the scalp. Each electrode in its specific location on the scalp produces a signal analogous to the currents it detects from the nerves local to it inside the brain. The electrode location information is also useful because that location and the corresponding collective electrode signals from that part of the brain, when combined with signals from other electrodes provides information about the activity of the brain. To assist the user in placing the electrodes consistently, templates or harnesses are often used.

In other applications, for example, electroencephalograph testing (EEG) and intraoperative monitoring (IOM), however, multiple electrodes may be used on other parts of the body to derive information about the condition of the patient. In these applications the positioning of the electrodes with respect to each other is also as important as the information sensed by each electrode.

Better placement of the electrodes in this latter instance would be advantageous.

### SUMMARY

According to its major aspects and briefly recited, disclosed herein is a multi-electrode set that may be used as a unit to preserve consistent and appropriate inter-electrode spacing and thereby facilitate application of the electrodes to the patient but also generate information that is more consistent.

A feature of the disclosure is a carrier having a first side and an opposing second side. The second side has one or more recesses formed in it, arranged in pre-selected locations. The recesses carry electrodes so that, when the second side of the carrier is applied to a surface, the electrodes will be in engagement with that surface in their pre-selected locations.

Another feature of the disclosure is that an adhesive, protected by a peel-off covering, holds the carrier to the patient's body at the point where it is applied.

Another feature of the disclosure is that each electrode may be carried in its recess by an electrode applicator.

Still another feature of the disclosure is that each electrode may be releasably carried in its recess by an electrode applicator.

Yet another feature of the disclosure is that each electrode may be releasably carried in its recess by an electrode applicator with a removeable cap.

Still another feature of the disclosure is an electrical conductor carried by the carrier. These electrical conductors have a first end and a second end. The first end of the electrical conductor is connected to the electrode. The second end of the electrical conductor may be connected to a multi-pin connector to facilitate plugging the electrical conductor into a monitoring device.

Another feature of the disclosure is that the carrier may be flat or curved.

Still other features of the disclosure are that the electrode carried in the recess of the carrier may be self-embedding, may be a stimulating- or a recording-type electrode, and may be used as a ground conductor or a reference conductor.

Other features and their advantages will be apparent to those skilled in the neurological monitoring and stimulating arts from a careful reading of the Detailed Description accompanied by the following drawings

### BRIEF DESCRIPTION OF THE DRAWINGS

In the figures,

FIG. 1 is a perspective view of one side of a multi-electrode array carrier with the electrical connectors attached via the carrier to a multi-pin connector, according to an aspect of the disclosure;

FIG. 2 is a perspective view of an opposing side of a multi-electrode array carrier shown in FIG. 1 with embedding electrodes visible in the carrier, according to an aspect of the disclosure;

FIG. 3 is a multi-electrode array carrier for use on an arm with the electrical connectors attached to a multi-pin connector, according to an aspect of the disclosure; and

FIG. 4 is a cross sectional view of a portion of the multi-electrode array carrier of FIG. 1, according to an aspect of the disclosure.

### DETAILED DESCRIPTION

The present disclosure teaches a multi-electrode set in which a carrier is used to hold several electrodes together in a fixed relationship, array or matrix not only prior to application but subsequent to application of it to a patient's body. The electrode array carrier has an adhesive applied to one side so that all of the electrodes in the array may be brought into engagement with the skin surface at once, and then remain adhered to the skin during use.

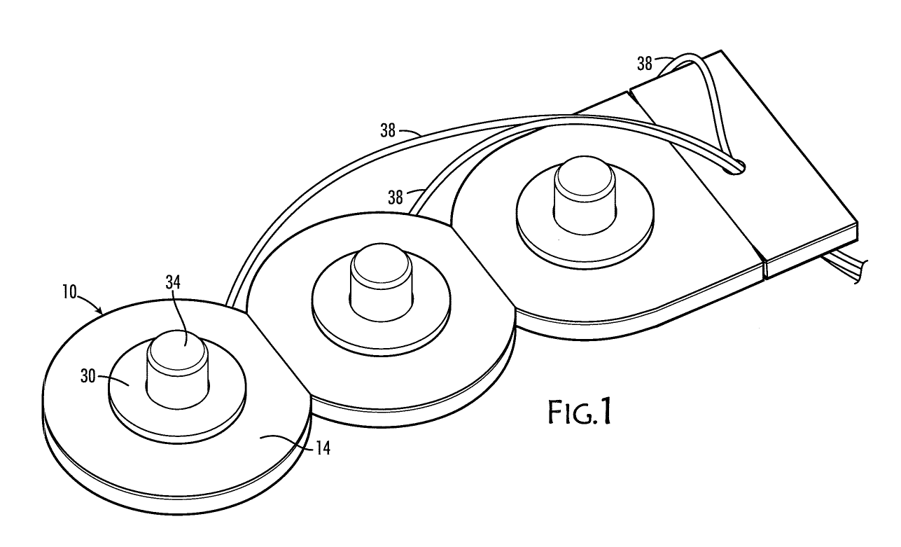

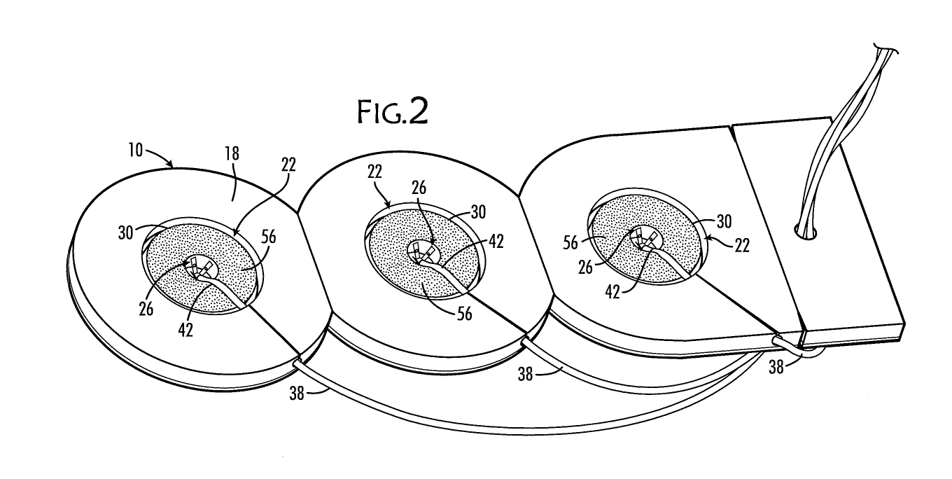

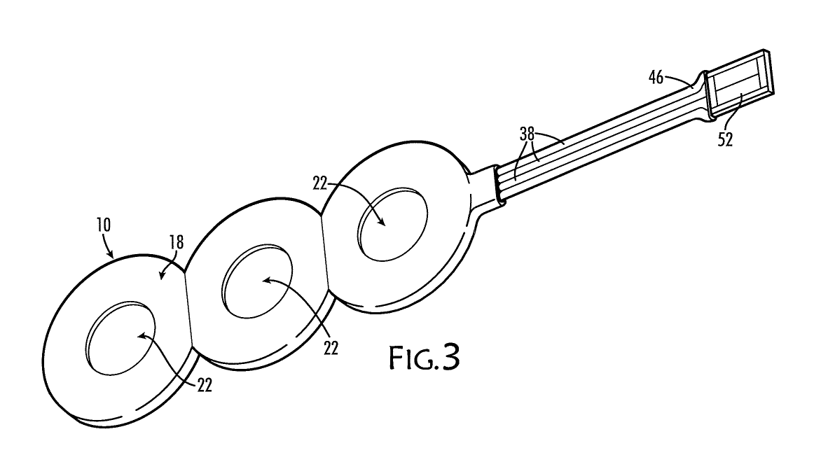

The carrier 10, shown in FIGS. 1-4, has a first side 14 and an opposing second side 18. Second side 18 has plural recesses 22 formed in it, arranged in preselected locations. Plural recesses 22 carry plural electrodes 26. As shown, each recess 22 may carry an electrode 26 so that, when second side 18 of carrier 10 is applied to a skin surface, electrode 26 will be in engagement with that surface and held in its preselected location with respect to other electrodes 26 held by carrier 10. Each electrode 26 may be carried in its recess 22 by an electrode applicator 30, which may have a removable cap 34.

An electrode applicator 30 is a device that holds an electrode 26 safely, prior to use, so as to prevent it from becoming contaminated, damaged or causing injury. When carrier 10 is in position, electrode 26 is ejected from its electrode applicator 30, such as by pressing a button underneath removable cap 34, and as taught in U.S. Pat. No. 9,480,412, for a disposable electrode assembly, assigned to Rhythmlink International LLC. Removable cap 34 prevents accidental ejection of electrode 26.

Plural electrodes 26 may also include plural electrical conductors 38. As shown, each electrode 26 may have an electrical conductor 38, typically a wire, that is also carried by carrier 10. Multiple electrodes 26 may have their individual electrical conductors brought together through carrier 10 for convenience and may form a ribbon cable. Electrical conductors 38 have a first end 42 and a second end 46. First end 42 of electrical conductor 38 is connected to electrode 26 in recess 22. Second end 46 of electrical conductor 38 may be connected to a multi-pin connector 52 to facilitate electrical connection of several electrical conductors 38 to a monitoring device not shown.

An adhesive 56 is applied to second side 18 of carrier 10 around recesses 22 so that, when second side 18 of carrier 10 is applied to the skin surface, adhesive 56 on second side 18 causes carrier 10 to stick to that skin surface, and to hold electrode 26 in engagement with that surface for the duration of attachment. A covering 60, such as a release paper, may be applied over adhesive 56 prior to use so that carrier 10 does not inadvertently become attached to another surface but, only when ready and positioned for use, the covering 60 is removed and adhesive 56 is exposed, carrier 10 can then be adhered to the skin surface.

Carrier 10 may be flat or curved to conform to the surface to which it will be attached. Thus, if the shape of the surface is level without marked projections or depressions, or instead has marked projections or depressions, the carrier 10 may be shaped complement the surface shape.

The type of electrode 26 may be self-embedding, stimulating, or recording, and may be used as a ground conductor or a reference conductor. Electrodes 26 used with the present carrier that are self-embedding type may be as disclosed in U.S. Pat. No. 8,428,681, which are manufactured and sold under the trademark PRESS-ON by Persyst Development Corporation.

The number of electrodes 26 carried by carrier 10 and their configuration and spacing will depend on the application. FIGS. 1-3 show multi-electrode arrays, including three electrode arrays in a linear array, which may be useful, for example, when performing neurological monitoring or testing on an arm. One of electrodes 26 may be a ground electrode, at ground potential, a second may be a reference electrode, used for obtaining background currents, and the third may be over the nerve being researched to capture currents at that location, which can then be compared to the second electrode's signals for reference and the ground position to determine if the signals are positive or negative. These electrodes may be in a linear array because that arrangement may be more natural for a neurological study of a structure shaped like an arm.

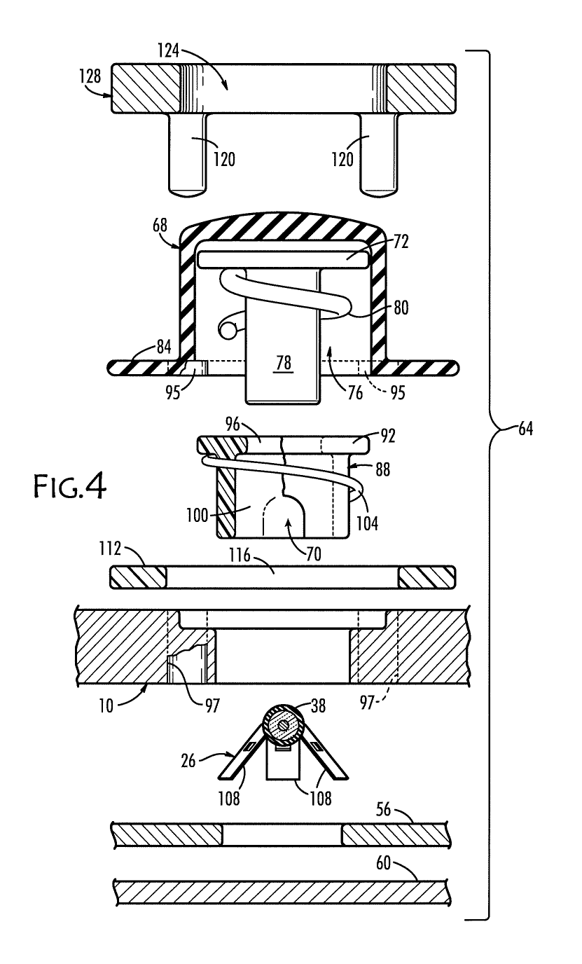

FIG. 4 shows an exploded view of a portion of the multi-electrode array carrier 64 that shows aspects of the electrode applicator 30. Button 68 receives a first plunger 72 in its interior 76. A first spring 80 having a first spring force engages a top flange 84 of first plunger 72. A second plunger 88, also with a flange 92 but with a hole 96 formed in flange 92, is received in interior 76 of button 68, so that first plunger 72 penetrates through hole 96 of flange 92. Second plunger 88 has a hollow interior 100 that will receive electrode 26 when electrode 26 is loaded into electrode applicator 30.

Second plunger 88 has a second spring 104 with a different spring force. Both first and second springs 80, 104, resist pressure on button 68 but the first spring force of first spring 80 is greater than the second spring force of second spring 104 so that force on button 68 first presses second plunger 88 down and then, when the resistance against further movement of second plunger 88 has increased sufficiently, first plunger 72 will be moved against the first spring force of first spring 80, until electrode 26 is ejected from electrode applicator 30.

Carrier 10 protects legs 108 of electrode 26 and recess 22 has an axis parallel to that of first and second plungers 72, 88. Flange 84 of button 68 also has plural circular holes 95 that may be placed in registration with carrier holes 97 of carrier 10. An alignment ring 112 having a central hole 116 surrounds button 68 and carries plural depending posts 120 that are axially aligned, dimensioned and deployed to fit into holes 97 of carrier 10 and holes 95 of flange 84 of button 68, thereby aligning and maintaining the alignment of button 68, including its flange 92, and carrier 10 before, after and during operation to release electrode 26.

In operation, an electrode 26 may be loaded into interior 100 of second plunger 88 so that its electrical conductor 38 extends through a channel 124 in carrier 10. After covering 60 is removed, carrier 10 is positioned against the skin of a patient. The user next applies steady pressure on button 68 against first and second springs 80, 104, respectively. Second spring 104, with a smaller spring force than that of first spring 80, compresses more readily than first spring 80. When top flange 84 of second plunger 88 reaches the end of its travel, continued pressure on button 68 no longer compresses second spring 104 but begins to compress first spring 80, thereby moving first plunger 72 toward retention ring 62. When first plunger 72 travels sufficiently far inside of second plunger 88, it reaches electrode 26 expelling it from carrier 10, although carrier 10 remains attached to the skin of the patient because of adhesive 56.

Those skilled in the art of neurological research and monitoring will appreciate many modifications and substitutions may be made in the foregoing description of embodiments of the disclosed stick-on, multi-electrode array without departing from the spirit and scope of the disclosure.

What is claimed is:

1. A device, comprising:
(a) a carrier having a first side and an opposing second side, said second side having a recess formed therein;
(b) an electrode releasably held by an electrode applicator, said electrode applicator carried in said recess, and operable to release said electrode when said second side of said carrier is applied to a surface, said electrode is operable to engage said surface;
(c) an electrical conductor carried by said carrier having a first end and a second end, said first end of said electrical conductor being connected to said electrode; and
(d) an adhesive applied to said second side of said carrier so that, when said second side of said carrier is applied to said surface, said adhesive on said second side of said carrier is operable to stick said carrier to said surface.

2. The device of claim 1, wherein said electrode applicator includes a removeable cap.

3. The device of claim 1, wherein said electrical conductor is a ground conductor.

4. The device of claim 1, wherein said electrical conductor is a reference conductor.

5. The device of claim 1, wherein said electrode is a self-embedding electrode.

6. The device of claim 1, further comprising a covering over said adhesive on said second side, wherein said covering is releaseable from said adhesive, and wherein said adhesive is exposed and operable to adhere said second side to said surface when said covering is removed.

7. The device of claim 1, further comprising a release paper on said adhesive on said second side, said release paper being releaseable from said adhesive.

8. A device, comprising:
(a) a carrier having a first side and an opposing second side, said second side having plural recesses formed therein;
(b) an electrode applicator;
(b) plural electrodes carried by said carrier, an electrode of said plural electrodes held by said electrode applicator in a recess of said plural recesses, said electrode applicator is operable to release said electrode when said second side of said carrier is applied to a surface, said electrode is operable to engage said surface;
(c) plural electrical conductors carried by said carrier, an electrical conductor of said plural electrical conductors having a first end and a second end, said first end of said electrical conductor connected to said electrode of said plural electrodes; and
(d) an adhesive applied to said second side of said carrier so that, when said second side of said carrier is applied to said surface, said adhesive on said second side to is operable to stick to said surface, and wherein said electrode of said plural electrodes is operable to engage with said surface.

9. The device of claim 8, further comprising a covering over said adhesive on said second side, wherein said covering is releasable from said adhesive, and wherein said adhesive is operable to adhere to said surface when said covering is removed from said second side.

10. The device of claim 8, further comprising a release paper on said adhesive on said second side, said release paper being releaseable from said adhesive, wherein said adhesive is exposed and is operable to said surface.

11. The device of claim 8, wherein said electrode of said plural electrodes is releasable from said applicator in said recess.

12. The device of claim 8, wherein said plural recesses are arranged in preselected positions on said carrier.

13. The device of claim 8, wherein said surface is flat, and wherein said carrier is flat to conform to said surface.

14. The device of claim 8, wherein said applicator includes a removeable cap, and wherein said electrode of said plural electrodes is operable to be attached to a surface.

15. The device of claim 8, wherein said second end of said electrical conductor of said plural electrodes is operable to be joined to a connector.

16. The device of claim 8, wherein said electrical conductor of said plural electrode conductors is a ground conductor.

17. The device of claim 8, wherein said electrical conductor of said plural electrode conductors is a reference conductor.

18. The device of claim 8, wherein said electrode of said plural electrodes is a self-embedding electrode.

19. The device of claim 8, wherein said electrode is a stimulating electrode.

20. The device of claim 8, wherein said electrode is a recording electrode.
